(19) United States
(12) Patent Application Publication
(10) Pub. No.: US 2026/0080989 A1
(43) Pub. Date: Mar. 19, 2026

(54) ULTRASOUND EXAMINATION METHODS, APPARATUS AND ULTRASOUND EXAMINATION SYSTEMS

(71) Applicant: SHENZHEN MINDRAY BIO-MEDICAL ELECTRONICS CO., LTD., Shenzhen (CN)

(72) Inventors: Jianhong GAO, Shenzhen (CN); Wei GUO, Shenzhen (CN); Baohui SUN, Shenzhen (CN); Jiaoni LI, Shenzhen (CN); Song HAN, Shenzhen (CN)

(73) Assignee: SHENZHEN MINDRAY BIO-MEDICAL ELECTRONICS CO., LTD., Shenzhen (CN)

(21) Appl. No.: 19/330,990

(22) Filed: Sep. 17, 2025

(30) Foreign Application Priority Data
Sep. 19, 2024 (CN) 202411311191.3

(51) Int. Cl. G16H 10/60; G16H 15/00; G16H 30/20; G16H 50/30; G16H 70/00

(52) U.S. Cl. CPC G16H 10/60; G16H 15/00; G16H 30/20; G16H 50/30; G16H 70/00

(57) ABSTRACT

The present application provides an ultrasound examination method, apparatus and ultrasound examination system, which belongs to the technical field of ultrasound examination. The method includes: obtaining an ultrasound examination task for a patient through an ultrasound workstation system; transmitting the ultrasound examination task to an ultrasound device; receiving ultrasound data corresponding to the ultrasound examination task returned from the ultrasound device; obtaining an examination result that is inputted by a user based on the ultrasound data; and transmitting the ultrasound data and the examination result to the ultrasound workstation system.

### CROSS-REFERENCE TO RELATED APPLICATION

The present application claims priority to Chinese Patent Application No. 202411311191.3, filed on Sep. 19, 2024, the disclosure of which is hereby incorporated by reference in its entirety.

### BACKGROUND

Ultrasound workstations are commonly used in hospitals to manage examination images and generate ultrasound examination reports. However, since workstations in different hospitals are typically products from different manufacturers, their workstation systems often provide only basic, general functions such as image management and report generation. For specialized ultrasound examinations, particularly in fields like obstetrics where specific tasks (e.g., fetal growth parameter measurements) are performed, the necessary specialized functions are lacking. Consequently, manual referencing or the use of third-party tools is required, leading to lower quality and efficiency in specialized ultrasound examinations.

### SUMMARY

The present application relates to ultrasound examination, particularly to ultrasound examination methods, apparatus and ultrasound examination systems.

Embodiments of the present application provide ultrasound examination methods, apparatus and systems to improve the quality and efficiency of specialized ultrasound examinations.

According to a first aspect, an ultrasound examination method provided in some embodiments of the present disclosure is adapted for use with a specialized software system, wherein the specialized software system provides specialized functions for specialized ultrasound examinations, and the specialized software system is connected to the ultrasound workstation system in the form of middleware; and the method may comprise:
obtaining the ultrasound examination task for a patient via the ultrasound workstation system;
transmitting the ultrasound examination task to an ultrasound device;
receiving ultrasound data corresponding to the ultrasound examination task from the ultrasound device;
obtaining an examination result that is inputted by a user based on the ultrasound data; and
transmitting the ultrasound data and the examination result to the ultrasound workstation system.

According to a second aspect, an ultrasound examination method provided in some embodiments of the present disclosure is adapted for use with a computer device, wherein the computer device comprises a specialized software system and an ultrasound workstation system, the specialized software system provides specialized functions for specialized ultrasound examinations, and the specialized software system is connected to the ultrasound workstation system in the form of middleware; and the method may comprise:
the ultrasound workstation system opening a patient report;
the specialized software system obtaining a ultrasound examination task for a patient based on the patient report;
the specialized software system transmitting the ultrasound examination task to an ultrasound device;
the specialized software system receiving ultrasound data that corresponds to the ultrasound examination task and is returned from the ultrasound device;
the specialized software system obtaining an examination result that is inputted by a user based on the ultrasound data; and
the ultrasound workstation system receiving the ultrasound data and the examination result transmitted by the specialized software system.

According to a third aspect, a computer device provided in some embodiments of the present disclosure may comprise:
one or more processors, configured to work individually or jointly to execute:
the steps of the ultrasound examination method according to any one of the embodiments of the present application; or
the steps of the ultrasound examination method according to any one of the embodiments of the present application.

According to a fourth aspect, an ultrasound examination system provided in some embodiments of the present disclosure may comprise:
an ultrasound device; and
a computer device according to any one of the embodiments of the present application, wherein the computer device is connected to the ultrasound device.

The ultrasound examination method provided in the embodiments of the present application is used for a specialized software system, which provides specialty-specific clinical ultrasound examination functions in a medical specialty; the ultrasound examination method includes: obtaining an ultrasound examination task for a patient through an ultrasound workstation system; transmitting the ultrasound examination task to an ultrasound device; receiving ultrasound data corresponding to the ultrasound examination task returned from the ultrasound device; obtaining an examination result that is inputted by a user based on the ultrasound data; and sending the ultrasound data and the examination result to the ultrasound workstation system. In the embodiments of the present application, the specialized software system is connected to the ultrasound workstation system for use in the form of middleware, enabling the ultrasound examination method to provide specialty-specific clinical ultrasound examination functions without replacing existing ultrasound workstations. This assists in performing specialty-specific ultrasound examinations, thereby significantly improving the quality and efficiency of such examinations.

### BRIEF DESCRIPTION OF THE DRAWINGS

FIG. 1 is a schematic flowchart diagram illustrating steps of an ultrasound examination method according to some embodiments of the present application;

FIG. 2 is a diagram showing an application scenario of the ultrasound examination method according to some embodiments of the present application;

FIG. 3 is a diagram showing another application scenario of the ultrasound examination method according to some embodiments of the present application;

FIG. 4 is a showing a further application scenario of the ultrasound examination method according to some embodiments of the present application;

FIG. 5 is a schematic diagram illustrating a scenario for implementing the ultrasound examination method according to some embodiments of the present application;

FIG. 6 is a schematic flowchart diagram illustrating steps of another ultrasound examination method according to some embodiments of the present application;

FIG. 7 is a schematic block diagram of a computer device according to some embodiments of the present application; and

FIG. 8 is a schematic block diagram of an ultrasound examination system according to some embodiments of the present application.

### DETAILED DESCRIPTION

The technical solutions of the embodiments of the present application will be described below clearly and completely with reference to the accompanying drawings in the embodiments of the present application. It is apparent that the described embodiments represent only a portion of the embodiments of the present application, rather than all possible embodiments. All other embodiments derived by those skilled in the art based on the embodiments herein without creative effort shall fall within the scope of protection sought by the present application.

The flowcharts depicted in the accompanying drawings represent exemplary embodiments are only, and need not include all possible content and operations/steps, nor must they be executed in the described order. For instance, certain operations/steps may be divided, combined, or partially consolidated. Consequently, the actual execution sequence may vary based on practical circumstances.

It should be understood that the terminology employed herein is for the purpose of describing particular embodiments only and is not to be construed as limiting the application. As used the specification and claims of this application: the singular form encompass plural referents unless the context explicitly dictates otherwise, and the terms “a”, “an” and “the” are inclusive of plural interpretations where context permits.

At present, the ultrasound workstations used in different hospitals are from different manufacturers. To solve the problems encountered in specialized fields such as obstetric ultrasound examinations and meet the professional demands of obstetric exams, an overall replacement of the ultrasound workstation system is necessary. However, this overall replacement requires integration with various information systems such as the hospital information system (HIS) and electronic medical record system (EMR) involving not only different departments such as the ultrasound department and the information technology (IT) department but also presenting challenges such as high investment, long decision-making cycles, and prolonged construction periods. Therefore, the overall replacement of the ultrasound workstation system is highly difficult.

Traditional ultrasound workstation systems can only provide basic, general functions such as image management and report generation management. They lack specialized functions for specialized fields such as obstetric ultrasound examinations. As a result, the difficulties and problems existing in obstetric ultrasound examinations are not resolved, many clinical demands remain unmet, and the quality and efficiency of specialized ultrasound examinations are low.

For instance, traditional ultrasound workstations used for obstetric ultrasound examinations lack the following specialized functions:
1) The large number of standard sections required for scanning in obstetric examinations makes it challenging to ensure their quality and completeness. Due to the lack of real-time quality control for sections during examination, ensuring section quality and completeness relies solely on the physicians'experience and skill.
2) Different hospitals use different fetal growth reference standards, and some of these standards are relatively new. The ultrasound device and workstations lack the department's preferred reference standards. Doctors need to use application software or consult paper reference charts to compare examination results, which is inconvenient and cumbersome.
3) Many ultrasound workstations lack fetal growth curves, impeding doctors' ability to accurately assess fetal growth. This necessitates searching for historical examination records to facilitate comparison and diagnosis.
4) Measurement results must be recorded manually, and anatomical structure results require manual editing in the report template. These manual process are time-consuming and error-prone.
5) Measurement data and examination results are not stored as structured data, hindering search and capabilities and failing to meet academic research requirements.

To address the current issues of suboptimal quality and efficiency in specialized ultrasound examinations, embodiments of the present application provide an ultrasound examination method, apparatus and ultrasound examination system. This solution can provide specialized functions for specialized ultrasound examinations without replacing existing ultrasound workstations, thereby assisting in specialized ultrasound examinations and significantly enhancing their quality and efficiency.

This ultrasound examination method can be applied to computer devices. Such computer devices may include mobile devices or servers, with the mobile devices being electronic devices such as smartphones, tablets, notebook computers, desktop computers, personal digital assistants, and wearable devices.

For example, the computer device may be an ultrasound examination room personal computer (PC). This PC is installed with a specialized software system and hosts an ultrasound workstation. The specialized software system is connected to the ultrasound workstation system in the form of middleware and provides specialized functions for specialized ultrasound examinations. Furthermore, the ultrasound workstation may be a component running on this PC.

The following describes some embodiments of the present application in detail with reference to the accompanying drawings. Where no conflict exists, the described embodiments and/or features thereof may be mutually combined.

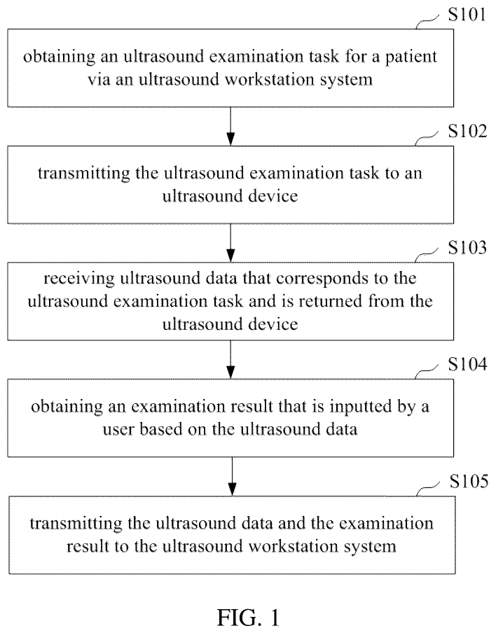

Please refer to FIG. 1, which is a schematic diagram illustrating the steps of an ultrasound examination method according to some embodiments of the present application.

As shown in FIG. 1, the ultrasound examination method comprises steps S101 through S105.

This ultrasound examination method is applied to a specialized software system which provides specialized functions for specialized ultrasound examination. The specialized software system is connected to the ultrasound workstation system in the form of middleware.

Step S101: obtaining an ultrasound examination task for a patient via an ultrasound workstation system.

The specialized software system functions as middleware, positioned between the operating system software and the ultrasound workstation system. It provides a runtime environment to enable specialized functions for specialized ultrasound examinations. Specialized fields may include medical disciplines such as obstetrics, cardiology, neurology, and oncology. Correspondingly, the specialized software systems may be, for example, obstetric ultrasound software systems.

For example, in the case where the specialized field is obstetrics, the specialized software system may include an obstetric ultrasound software system, which provides specialized ultrasound functions for obstetric ultrasound examinations. This specialized ultrasound examination functions in the obstetric field may include, among others, fetal growth parameters or fetal growth curves.

The ultrasound workstation system is an ultrasound workstation software. It supports various models of ultrasound devices and provides conventional ultrasound examinations. For instance, it integrates functional modules for patient registration, image acquisition, diagnostic editing, report printing, image post-processing, medical record queries, and data backup, thereby enabling these capabilities.

It should be noted that the specialized software system may be a browser or a client application, and may be installed and executed on the same computer device as the ultrasound workstation system. When the specialized software system is a browser, a Browser-Server architecture may be adopted. When the specialized software system is a client application, a Client-Server architecture may be adopted.

In an embodiment, the specialized software system may be connected to the ultrasound workstation system using a predefined first interface. The first interface may include, but is not limited to, an application programming interface (API) and network sockets.

It should be noted that when the specialized software system is connected to the ultrasound workstation system via the predefined first interface, it effectively integrates with the ultrasound workstation system. This integration enables seamless data interaction between the two systems, resulting in a smoother and more user-friendly experience.

In an embodiment, the specialized software system may be connected to the ultrasound workstation system using a predetermined first communication protocol. The first communication protocol may include, but is not limited to, the digital imaging and communications in medicine (DICOM) protocol.

It should be noted that when the specialized software system is connected to the ultrasound workstation system using the predetermined first communication protocol, this connection represents a non-integrated approach, yet still facilitates data interaction between the two systems while ensuring operational reliability.

It should be understood that since the embodiments of the present application can provide specialized functions for specialized ultrasound examinations without replacing the entire ultrasound workstation system, and the specialized software system is connected to the ultrasound workstation system as middleware, the specialized software system does not directly connect to the medical record database and needs to obtain the ultrasound examination task for the patient indirectly through the ultrasound workstation system. Here, the ultrasound examination task may refer to the relevant tasks requiring the use of the ultrasound device for conducting ultrasound examinations. For example, the ultrasound examination task may be a specialized ultrasound examination task in a specialized field, involving specialized functions for specialized ultrasound examinations.

In an embodiment, obtaining the ultrasound examination task for a patient via an ultrasound workstation system may include: receiving patient examination information sent by the ultrasound workstation system; and obtaining the patient's ultrasound examination task based on the patient examination information. Here, the patient examination information refers to information indicating the examination required for the current patient. For example, such information may specify that the current patient is scheduled for cardiac examination, obstetric examination, gynecological examination, abdominal examination, carotid artery examination, or thyroid examination; alternatively, it may indicate a contrast-enhanced examination, elastography examination, early/mid-pregnancy examination, NT screening; or, it may comprises any other information designating the required examination.

The patient examination information may be obtained by the ultrasound workstation system based on the current patient's relevant information (such as name, ID, etc., herein referred to as “patient information”; said patient information may be user-input, read from a patient report, or obtained through other means), for example, from the hospital's medical record database or other databases; alternatively, the patient report may contain the patient examination information, enabling the ultrasound workstation system to obtain it based on the current patient's report.

Since the patient examination information specifies the examination required for the current patient, after the ultrasound device receives the patient examination information, it can determine both the specific examination to be conducted and the corresponding ultrasound examination task to be performed for the current patient.

It should be noted that when the specialized software system is connected to the ultrasound workstation system via the predefined first interface, said system can directly receive the patient examination information transmitted by the ultrasound workstation system. Consequently, the specialized software system may derive the patient's ultrasound examination task from the patient examination information.

In an embodiment, obtaining the ultrasound examination task for a patient via the ultrasound workstation system may include: acquiring said task from a patient report when said report is opened in the ultrasound workstation system. As used herein, the patient report may refer to a patient report newly created for the current patient, or a historical report that is reopened during the current examination to function as the current examination report.

The acquisition of the ultrasound examination task for the patient from the patient report may include: capturing a screenshot of the patient report; identifying the patient's identification information from the screenshot content; and retrieving the ultrasound examination task for the patient from the task worklist based on said identification information.

It should be noted that when the specialized software system is connected to the ultrasound workstation system via a predetermined first communication protocol, it cannot directly receive the patient report transmitted by the ultrasound workstation system. Consequently, a screenshot of the patient report may be captured to identify the patient's identification information therefrom, enabling precise retrieval of the ultrasound examination task for the patient from the task worklist based on the patient's identification information.

Obtaining the ultrasound examination task for the patient based on the patient report may include: in response to a user-triggered query operation, querying the ultrasound examination task for the patient from the task worklist; wherein the query operation utilizes the patient's identification information reported in the patient report.

It should be noted that when the patient report is opened in the ultrasound workstation system, a user may manually initiate the query of the ultrasound examination task for the patient, thereby allowing the specialized software system to accurately retrieve said task.

Step S102: transmitting the ultrasound examination task to an ultrasound device.

Here, since the specialized software system is connected to the ultrasound device, the ultrasound examination task may be transmitted to it, enabling the ultrasound device to perform the corresponding ultrasound examination based on the task and generate associated ultrasound data.

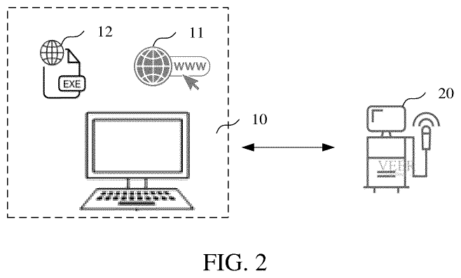

For example, as shown in FIG. 2, a specialized software system 11 and an ultrasound workstation system 12 are installed on a computer device 10. The computer device 10 is in communication with the ultrasound device 20 through a predetermined second communication protocol or a predefined second interface. After the specialized software system 11 acquires an ultrasound examination task for a patient through the ultrasound workstation system 12, it transmits the task to the ultrasound device 20, allowing the ultrasound device 20 to perform the corresponding ultrasound examination based on the task transmitted by the specialized software system 11 and generate associated ultrasound data.

In an embodiment, the patient information (as mentioned earlier) may also be obtained via the ultrasound workstation system and transmitted to the ultrasound device for use during the examination.

In an embodiment, the patient's historical examination data may also be obtained via the ultrasound workstation system and transmitted to the ultrasound device. In this way, when the ultrasound device is performing the examination task, it may use the historical examination data to generate corresponding statistical or analytical results, such as the fetal growth chart mentioned below.

Step S103: receiving ultrasound data that corresponds to the ultrasound examination task and is returned from the ultrasound device.

The ultrasound data corresponding to the ultrasound examination task from the ultrasound device may include ultrasound images, which reflect the differences in acoustic parameters in the medium. For example, when the ultrasound examination task includes the examination of various parts or organs of the fetus, the ultrasound images can depict various parts or organs of the fetus.

In an embodiment, the ultrasound data may include measurement data of a target object. The target object may be a human body part or organ depicted in the ultrasound image. For example, when the ultrasound examination task includes measuring growth parameters for various parts or organs of the fetus, the target object may be the target part or target organ of the fetus, and the measurement data in the ultrasound data may be the dimensional data of the target part or organ of the fetus.

In an embodiment, the measurement data in the ultrasound data is structured data. It should be noted that the ultrasound examination data recorded in the current ultrasound workstation system is not structured data, making it difficult to search and analyze and unable to meet the needs of scientific research. Therefore, in this embodiment, the measurement data in the ultrasound data is configured as structured data, thereby facilitating the search and analysis of the ultrasound data and meeting the needs of scientific research.

For example, as shown in FIG. 2, when the specialist software system 11 and the ultrasound device 20 communicate via the DICOM protocol, the specialist software system 11 transmits the ultrasound examination task to the ultrasound device 20, and the ultrasound device 20 returns the ultrasound data corresponding to the ultrasound examination task to the specialist software system 11. The ultrasound data includes ultrasound images and measurement data of the target object, and the measurement data of the target object may be in the DICOM structure reporting (SR) format.

In an embodiment, after receiving the ultrasound data corresponding to the ultrasound examination task returned from the ultrasound device, the method further includes: transmitting the ultrasound data to a server for storage; wherein the server includes at least one of: a hospital information system server, an electronic medical record system server, an ultrasound workstation system server, or a specialist software system server.

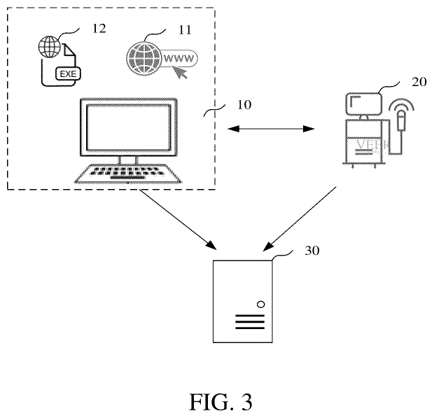

For example, as shown in FIG. 3, the specialist software system 11 is connected both to the ultrasound device 20 and to the ultrasound workstation server 30 via the DICOM protocol. When the specialist software system 11 receives the ultrasound images and DICOM SR from the ultrasound device 20, it forwards the ultrasound images and DICOM SR files to the ultrasound workstation server 30 using the DICOM protocol.

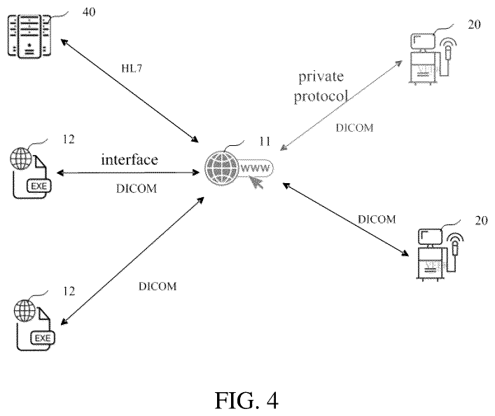

For example, as shown in FIG. 4, the specialist software system 11, acting as middleware software, is integrated with the ultrasound workstation system 12 via a predefined interface. Additionally, the specialist software system 11, acting as middleware software, is used in conjunction with the ultrasound workstation system 12 through the DICOM protocol. The specialist software system 11 can connect to the ultrasound devices 20 of the same manufacturer through both the DICOM protocol and a private protocol. Furthermore, the specialist software system 11 can connect to ultrasound devices 20 of different manufacturers through the DICOM protocol. Moreover, the specialist software system 11 is integrated with a server 40 via the HL7 protocol, where the server 40 includes the hospital information system server and the electronic medical record system server.

In an embodiment, the ultrasound data includes multiple ultrasound images. After receiving the ultrasound data corresponding to the ultrasound examination task returned by the ultrasound device, the method further includes: displaying an ultrasound image list containing the multiple ultrasound images; and determining an image to be printed from the multiple ultrasound images based on a selection operation performed on the ultrasound image list. Therefore, the user can view all the ultrasound images in the image list and select the image to be printed in the ultrasound examination report.

In an embodiment, the specialized software system includes an obstetric software system, and the ultrasound data includes at least one of an ultrasound image, fetal measurement data, and a fetal growth chart.

The obstetric software system provides obstetrics-specific functions for ultrasound examinations. When the obstetric software system acquires the ultrasound examination tasks of patients in the obstetric field through the ultrasound workstation system, the specialized software system may receive the ultrasound data from the ultrasound device that may include: ultrasound images of the uterine region, fetal measurement data, and/or fetal growth charts. No limitation is imposed herein.

For example, when the obstetric software system communicates with the ultrasound device via the DICOM protocol, and receives ultrasound data corresponding to the ultrasound examination task from the ultrasound device, the received ultrasound data includes ultrasound images and DICOM SR.

In an embodiment, the obstetric software system may connect to the ultrasound device through a predefined interface and transmit data via this interface, thereby simplifying the operations of the ultrasound device, and/or expanding and enhancing the functions of the ultrasound device, resulting in more user-friendly operation of the ultrasound device.

For instance, the obstetric software system may transmit the patient information, examination information, and gestational age data contained in the ultrasound examination task to the ultrasound device via the predefined interface. The ultrasound device may then automatically create an examination record for the patient, and display the patient information and examination information. Upon physician confirmation, the operator can commence image acquisition and perform clinical measurements on the ultrasound device.

In an embodiment, the ultrasound examination method further comprises: transmitting multiple sets of historical fetal measurement data to the ultrasound device, enabling the ultrasound device to generate a fetal growth chart based on the multiple sets of historical fetal measurement data, wherein the fetal growth chart serves to evaluate fetal growth.

The specialized software system transmits multiple sets of historical fetal measurement data to the ultrasound device. After the ultrasound device generates a fetal growth chart incorporating the multiple sets of historical fetal measurement data, physicians can directly evaluate fetal growth based on this curve at the ultrasound device and obtain relevant examination results, significantly improving the quality and efficiency of obstetric ultrasound examinations.

It should be noted that fetal growth reference standards vary across hospitals. Neither ultrasound devices nor ultrasound workstation systems incorporate clinical department-specific reference standards, necessitating manual comparison of examination results against mobile applications or paper-based growth parameter tables-an operationally cumbersome process. In the method of this embodiment, the specialized software system enables flexible configuration of fetal growth reference standards. For example, established fetal growth charts predetermined by hospitals may be configured as the current reference standard.

In an embodiment, the ultrasound examination method further comprises: setting a reference table for evaluating the standard fetal growth criteria in the specialized software system; and transmitting the reference table to the ultrasound device when the specialized software system is connected to the ultrasound device.

It should be noted that users may configure the reference table for evaluating the standard fetal growth criteria via the obstetric software system. When the obstetric software system is connected to the ultrasound device, the obstetric software system automatically transmits the reference table to the ultrasound device. Of course, a transmission instruction for the reference table may also be generated based on a user-triggered operation and transmitted to the ultrasound device.

By transmitting the reference table to the ultrasound device, doctors can use the reference table and the measured data during examinations on the ultrasound device to evaluate fetal growth status and make related diagnoses, thereby greatly improving the quality and efficiency of obstetric ultrasound examinations.

In an embodiment, the ultrasound examination method further includes: obtaining a formula calculation table for calculating the standard fetal growth criteria parameter values; and transmitting the formula calculation table to the ultrasound device.

Step S104: obtaining an examination result that is inputted by a user based on the ultrasound data.

After receiving the ultrasound data corresponding to the ultrasound examination task returned from the ultrasound device, users (doctors) may perform diagnoses based on the ultrasound data and input the examination results into the specialized software system. The specialized software system thus obtains the examination results that are inputted by the users based on the ultrasound data. Input methods for the examination results include but are not limited to: keyboard entry, mouse input, voice recognition. No limitations are imposed in the embodiments of the present application.

In an embodiment, the examination result is stored or managed in a structured data format. The ultrasound examination data recorded in current ultrasound workstation systems typically lacks structured formatting, making it difficult to search and analyze while failing to meet research requirements. Therefore, in the embodiments of the application, the ultrasound examination results are stored or managed as structured data, enabling efficient search and analysis of ultrasound data for research purposes.

For example, the specialized software system is connected to the ultrasound device via a predetermined second communication protocol. After the specialized software system receives the ultrasound images and DICOM SR from the ultrasound device, doctors input the examination results based on the ultrasound data in the specialized software system. The examination results may include measurement data of the target object, presence/absence of structural abnormalities.

For example, the specialized software system is connected to the ultrasound device via a predefined second interface. After performing diagnostic measurement on ultrasound scans acquired by the ultrasound device, doctors may input the examination results through a client application or a web-based interface. The examination results may include measurement data of the target object, determination of whether the measurement data is abnormal, and reasons for any detected abnormalities.

Step S105: transmitting the ultrasound data and the examination result to the ultrasound workstation system.

The ultrasound data may include user-selected ultrasound images and measurement data. The specialized software system may transmit the ultrasound data and the examination result to the ultrasound workstation system so that the ultrasound workstation system can insert the ultrasound data and the examination result into the patient report or save them to the medical record database.

In an embodiment, the examination result may be structured data. Before transmitting the ultrasound data and the examination result to the ultrasound workstation, the method also includes: analyzing the examination result into text format data.

It should be noted that when the examination result is structured data, it is necessary to parse the structured data to obtain text format data to facilitate review and confirmation by the ultrasound workstation system. For example, if the measurement data is a DICOM SR file, it needs to be parsed into a text measurement result so that the text measurement result can be automatically recorded in the patient's examination report, thereby facilitating interpretation by the patient and relevant doctors.

For example, the specialized software system is connected to the ultrasound workstation system via a predetermined first communication protocol. After transmitting the ultrasound data and the examination result to the ultrasound workstation system, the ultrasound data and examination result are inserted into the patient report on the ultrasound workstation system. After a doctor reviews and electronically signs to confirm, an ultrasound report is generated. Here, the ultrasound data may include ultrasound images and screenshots of measurement data.

For example, the specialized software system is connected to the ultrasound workstation system via a predefined first interface. The specialized software system sends ultrasound data, fetal growth charts, and examination results to the ultrasound workstation system. The specialized software system then automatically minimizes its own window and launches the ultrasound workstation system. Through the ultrasound workstation system, the following data is displayed: the ultrasound data, fetal growth charts, and examination results, etc. After the doctor reviews and signatures confirmation, an ultrasound report is generated.

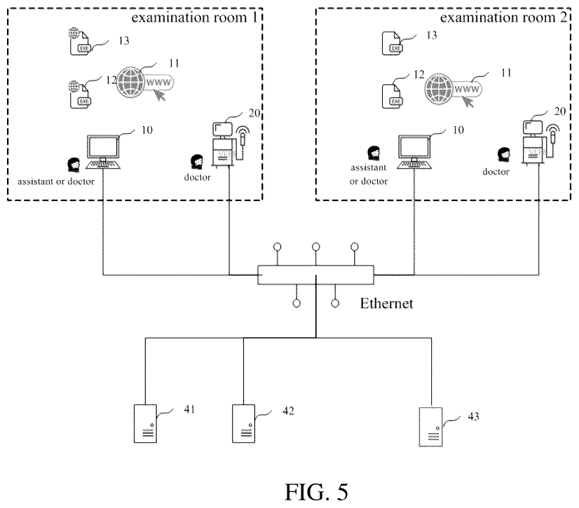

Please refer to FIG. 5, which is a schematic diagram of a scene for implementing the ultrasound examination method according to some embodiments of the present application. As shown in FIG. 5, a specialized ultrasound department includes an examination room 1 and an examination room 2. Both the examination rooms 1 and 2 are equipped with a computer device 10 and an ultrasound device 20. The computer device 10 is configured with a specialized software system 11, an ultrasound workstation system 12, and a calling system 13. The computer device 10 can be operated by assistant doctors, and the ultrasound device 20 can be operated by doctors. The computer devices 10 and the ultrasound devices 20 in the examination rooms 1 and 2 are respectively connected to a hospital information system server 41, an electronic medical record system server 42, and a specialized software system server 43 via Ethernet.

Specifically, the specialized software system 11 obtains an ultrasound examination task for a patient via the ultrasound workstation system 12 and transmits the ultrasound examination task to the ultrasound device 20. The ultrasound device 20, based on the ultrasound examination task transmitted by the specialized software system 11, performs corresponding scanning and measurement, thereby generating associated ultrasound data. The specialized software system 11 receives the ultrasound data corresponding to the ultrasound examination task from the ultrasound device 20. The specialized software system 11 obtains an examination result based on the ultrasound data input by a doctor. The specialized software system 11 transmits the ultrasound data and the examination result to the ultrasound workstation system 12.

The ultrasound examination method provided in the aforesaid embodiments is used for the specialized software system. The specialized software system provides specialized functions for specialized ultrasound examinations. The ultrasound examination method may include: obtaining an ultrasound examination task for a patient via the ultrasound workstation system; transmitting the ultrasound examination task to the ultrasound device; receiving ultrasound data corresponding to the ultrasound examination task from the ultrasound device; obtaining an examination result that is inputted by a user based on the ultrasound data; and transmitting the ultrasound data and the examination result to the ultrasound workstation system. In the embodiments of this application, the specialized software system is used in the form of middleware in connection with the ultrasound workstation system, enabling the ultrasound examination method to provide specialized functions for specialized ultrasound examinations without replacing the existing ultrasound workstation, thus greatly improving the quality and efficiency of specialized ultrasound examinations.

In the embodiments of this application, the specialized software system in the form of middleware can conveniently integrate with the existing ultrasound workstation system in a department. It can be installed and used in the department without replacing the entire ultrasound workstation system, thereby providing specialized functions for the ultrasound examinations in the specialized field. At the same time, it helps doctors address key challenges in specialized ultrasound examinations, improves diagnostic quality and efficiency, and provides structured reports to better support research and teaching. Additionally, the specialized software system can connect to the ultrasound device, enhancing its functions, simplifying operations, and making it more convenient and easier to use.

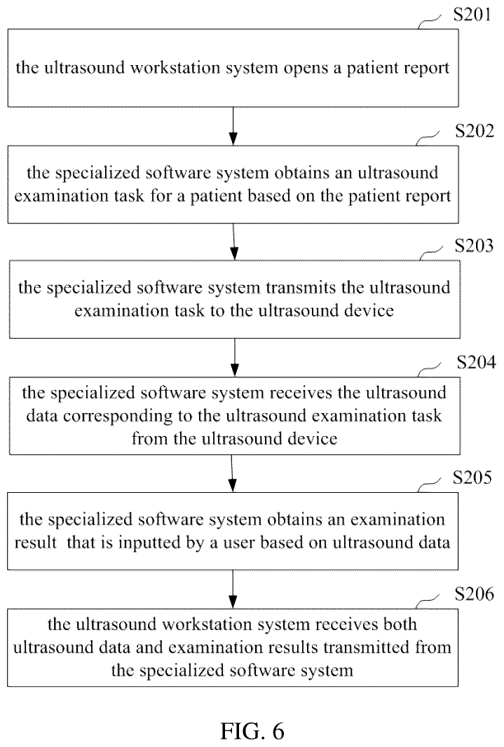

Please refer to FIG. 6, which illustrates a schematic flowchart of steps of another ultrasound examination method according to some embodiments of the present application.

As shown in FIG. 6, the ultrasound examination method comprises steps S201 through S206.

The shown ultrasound examination method is implemented on a computer device comprising a specialized software system and an ultrasound workstation system. The specialized software system provides specialized functions for specialized ultrasound examinations, and is connected to the ultrasound workstation system in the form of middleware.

The specialized software system is a middleware software, located between the operating system software and the ultrasound workstation system. The ultrasound workstation system is an ultrasound workstation software that provides conventional ultrasound examination management functions.

The specialized fields may include obstetrics, cardiology, neurology, oncology, and other medical fields. Corresponding specialized software systems may include, for example, obstetric software systems. For example, the specialized software system may include obstetric software systems, which provides specialized functions for ultrasound examinations in the obstetric field.

It should be noted that the specialized software system may be a browser or a client application and may be installed and run on the same computer device as the ultrasound workstation system. When the specialized software system is a browser, a browser-server architecture may be adopted. When the specialized software system is a client application, a client-server architecture may be adopted.

In an embodiment, the specialized software system is connected to the ultrasound workstation system through a predetermined first communication protocol or a predefined first interface. The first interface may include, but is not limited to, application programming interfaces (APIs) and network sockets. The first communication protocol may include, but is not limited to, digital imaging and communications in medicine (DICOM) protocol and private communication protocols.

It should be noted that the specialized software system and the ultrasound workstation system may be integrated, and they are connected via a predefined first interface, which may be defined in an interface database. Alternatively, they may not be integrated, and are connected via a predetermined first communication protocol. When the specialized software system is connected to the ultrasound workstation system via the first communication protocol or the first interface, the specific processing modes may be different.

Step S201: the ultrasound workstation system opens a patient report.

The ultrasound workstation system is connected to a medical record database and can obtain the patient report from the database. Accordingly, the patient report is opened by the ultrasound workstation system. In other words, the ultrasound workstation system obtains the patient report from the medical record database and opens it.

Step S202: the specialized software system obtains an ultrasound examination task for a patient based on the patient report.

Since the specialized software system acts as middleware, it does not directly connect to the medical record database but obtains the ultrasound examination task for the patient indirectly through the ultrasound workstation system.

In an embodiment, obtaining the ultrasound examination task for the patient based on the patient report comprises: capturing a screenshot of the patient report; identifying the patient's identification information from the screenshot content; and querying the ultrasound examination task for the patient from a task worklist based on the identification information. The ultrasound examination task may comprise tasks requiring an ultrasound device for ultrasound examinations.

It should be noted that when the specialized software system and the ultrasound workstation system are connected via a predetermined first communication protocol, the specialized software system cannot directly receive the patient report from the ultrasound workstation system. Therefore, it may capture a screenshot of the patient report, identify the patient's identification information from the screenshot content, and accurately query the ultrasound examination task for the patient from the task worklist based on this identification information.

In an embodiment, the patient report includes patient examination information. The obtaining of the ultrasound examination task for the patient based on the patient report comprises: receiving the patient examination information provided (called) by the ultrasound workstation system; and obtaining the ultrasound examination task for the patient based on the patient examination information.

It should be noted that when the specialized software system and the ultrasound workstation system are connected via a predefined first interface, the specialized software system can directly receive the patient examination information from the ultrasound workstation system, thereby enabling it to obtain the ultrasound examination task for the patient from the patient examination information.

In an embodiment, the obtaining of the ultrasound examination task for the patient based on the patient report comprises: querying the ultrasound examination task for the patient from the task worklist based on a query operation triggered by a user, wherein this query operation is performed based on the patient's identification information on the patient report.

It should be noted that when the ultrasound workstation system opens the patient report, the user may manually query the ultrasound examination task for the patient, enabling the specialized software system to obtain it accurately.

Step S203: the specialized software system transmits the ultrasound examination task to the ultrasound device.

The specialized software system is also in communication with the ultrasound device, allowing it to transmit the ultrasound examination task to the ultrasound device, thereby enabling the ultrasound device to perform corresponding ultrasound examinations based on the task and generate associated ultrasound data.

In an embodiment, the specialized software system and the ultrasound workstation system are installed on a computer device which is in communication with the ultrasound device via a predetermined second communication protocol or a predefined second interface. After obtaining the ultrasound examination task for the patient through the ultrasound workstation system, the specialized software system transmits the task to the ultrasound device. This enables the ultrasound device to perform corresponding ultrasound examinations based on the task transmitted by the specialized software system, thereby generating associated ultrasound data.

The second interface may include, but is not limited to, an application programming interface (API) and network sockets. The second communication protocol may include, but is not limited to, the digital imaging and communications in medicine (DICOM) protocol and private communication protocols.

In an embodiment, the specialized software system may also obtain patient information or the patient's historical examination data and transmit it to the ultrasound device.

Step S204: the specialized software system receives the ultrasound data corresponding to the ultrasound examination task from the ultrasound device.

The corresponding ultrasound data returned from the ultrasound device may include ultrasound images, which represents variations in acoustic parameters within the medium. The ultrasound data may also include measurement data of the target object. The target object may be a human body part or organ reflected in the ultrasound images, and the measurement data may be, for example, dimensional data of the target object.

In an embodiment, the measurement data and the examination result in the ultrasound data are structured data. This structured data may be formatted as, for example, a DICOM SR file. The use of structured data facilitates searching and analyzing ultrasound data, supporting research and educational requirements.

In an embodiment, after receiving the corresponding ultrasound data associated with the ultrasound examination task from the ultrasound device, the method further includes: transmitting the ultrasound data to a server for storage; wherein the server includes at least one of: a hospital information system server, an electronic medical record system server, an ultrasound workstation system server, and a specialized software system server. By sending the ultrasound data to the server for storage, the storage reliability of the ultrasound data can be improved.

In an embodiment, the ultrasound data comprises a plurality of ultrasound images; and after receiving the ultrasound data associated with the ultrasound examination task from the ultrasound device, the method further comprises: displaying an ultrasound image list containing the plurality of ultrasound images; based on a selection operation on the ultrasound image list, determining an image to be printed from the plurality of ultrasound images. Consequently, users can view all the ultrasound images through the image list and select the image to be printed in the ultrasound examination report.

In an embodiment, the specialized software system comprises an obstetric software system; and the ultrasound data includes at least one of: an ultrasound image, fetal measurement data, and a fetal growth chart.

The obstetric software system provides specialized functions for obstetric ultrasound examinations. When the obstetric software system acquires the ultrasound examination tasks for patients in the obstetric field through the ultrasound workstation system, the ultrasound data received by the specialized software system may include ultrasound images of the uterine region, fetal measurement data, or fetal growth charts, etc. No limitations are imposed in the embodiments of the present application.

It should be noted that the obstetric software system connects to the ultrasound device using a predefined interface and transmit data via the predefined interface, thus simplifying the operation of the ultrasound device, or extending its functionality, thereby improving operational efficiency and user experience.

In an embodiment, the ultrasound examination method further comprises: the specialized software system transmitting multiple sets of historical fetal measurement data to the ultrasound device, enabling the ultrasound device to generate a fetal growth chart based on the multiple sets of historical fetal measurement data. The fetal growth chart serves for fetal growth assessment.

It should be noted that fetal growth reference standards vary across hospitals. Since the ultrasound device and the ultrasound workstation systems lack institution-specific growth reference standards, and it is necessary to use mobile application software or paper-based growth parameter tables to compare the examination results, resulting in cumbersome workflows. In the embodiments of this application, the specialized software system enables dynamic configuration of the fetal growth reference standards. For example, a hospital-predefined fetal growth chart may be set as the reference standard for current examinations.

The specialized software system transmits the multiple sets of historical fetal measurement data to the ultrasound device. The ultrasound device is capable of plotting a fetal growth chart containing the multiple sets of historical fetal measurement data, thus enabling doctors to directly evaluate fetal growth based on the fetal growth chart on the ultrasound device and obtain associated examination results. This greatly improves the quality and efficiency of obstetric ultrasound examinations. In an embodiment, the ultrasound examination method further includes: the specialized software system acquiring a reference table for evaluating the standard fetal growth criteria; and the specialized software system, when connected to the ultrasound device, transmitting the reference table to the ultrasound device.

It should be noted that users can configure the reference table for evaluating standard fetal growth criteria on the obstetric software system. When the obstetric software system establishes a connection with the ultrasound device, it may automatically transmit the reference table to the ultrasound device. Alternatively, transmitting of the reference table may be triggered by a doctor's manual operation, wherein a transmission instruction is generated to transmit the reference table to the ultrasound device.

Following the transmission of the reference table to the ultrasound device, doctors can utilize both the reference table and the measured data during the ultrasound examination to evaluate fetal growth and conduct related diagnoses. This approach significantly enhances the quality and efficiency of obstetric ultrasound examinations.

Step S205: the specialized software system obtains an examination result that is inputted by a user based on ultrasound data.

After receiving the task-corresponding ultrasound data returned by the ultrasound device, the specialized software system enables doctors to formulate associated diagnoses based on such ultrasound data and input examination results therein. Consequently, the specialized software system can obtain the examination results based on ultrasound data input by the users (doctors).

For example, the specialized software system is connected to the ultrasound device via a predetermined second communication protocol. After the specialized software system receives the ultrasound images and measurement data of the target object transmitted by the ultrasound device, doctors can input the examination results based on the ultrasound data into the specialized software system. These examination results may include, but are not limited to: screenshots of the measurement data, determination of whether the measurement data is abnormal, and reasons for any detected abnormalities.

Step S206: the ultrasound workstation system receives both ultrasound data and examination results transmitted from the specialized software system.

Herein, the ultrasound data may comprise user-selected ultrasound images and measurement data. The specialized software system transmits the ultrasound data and examination results to the ultrasound workstation system, thereby enabling the ultrasound workstation system to integrate the ultrasound data and examination results into patient reports, or store them in a medical record database.

In an embodiment, the examination results are configured as structured data. Before transmitting the ultrasound data and examination results to the ultrasound workstation system, the method further includes: analyzing the examination results into text format data.

It should be noted that when the examination results are structured data, it is necessary to parse the structured data to obtain text format data. This conversion enables validation and confirmation via the ultrasound workstation system, or integration into patients' examination report, thereby rendering the results interpretable by both the patients and attending doctors.

In an embodiment, the ultrasound examination method further includes: the ultrasound workstation system inserting the ultrasound data and examination results into the patient report. It should be noted that after the ultrasound workstation system receives the ultrasound data and examination results transmitted from the specialized software system, it can copy the ultrasound data and examination results into the patient report, thereby achieving the update of the patient report and facilitating interpretation by the patients and attending doctors.

In an embodiment, the ultrasound examination method further includes: the ultrasound workstation system uploading the patient report to the electronic medical record system when the patient report has been approved. It should be noted that when the patient report has been approved, it can be uploaded to the electronic medical record system through the ultrasound workstation system, thereby achieving the secure storage of the patient report.

The ultrasound examination method provided by the above embodiments is used for a computer device, which includes a specialized software system and an ultrasound workstation system. The specialized software system provides specialized functions for ultrasound examinations in a specific field. The specialized software system is connected to the ultrasound workstation system in the form of middleware. The ultrasound examination method includes: opening a patient report by the ultrasound workstation system; obtaining ultrasound examination task for the patient through the patient report by the specialized software system; transmitting the ultrasound examination task to the ultrasound device by the specialized software system; receiving, by the specialized software system, ultrasound data corresponding to the ultrasound examination task returned by the ultrasound device; obtaining, by the specialized software system, examination results based on the ultrasound data input by a user; and receiving, by the ultrasound workstation system, the ultrasound data and examination results sent by the specialized software system. In the embodiments of this application, the specialized software system is connected and used as middleware with the ultrasound workstation system. This configuration enables the ultrasound examination method to provide specialized functions for ultrasound examinations in a specific field without replacing an existing ultrasound workstation system, facilitates assistance in specialized ultrasound examinations, and thereby significantly improves quality and efficiency of specialized ultrasound examinations.

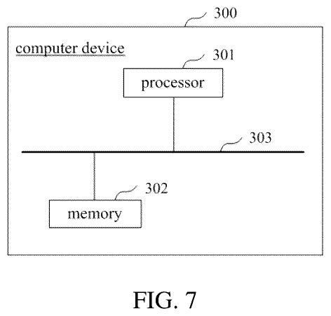

Please refer to FIG. 7, which is a schematic block diagram of a computer device provided in some embodiments of the present application.

As shown in FIG. 7, the computer device 300 includes one or more processors 301, operating independently or jointly to perform the steps of the ultrasound examination method as described in any embodiment of the present application.

Specifically, the processor 301 may be a microcontroller unit (MCU), a central processing unit (CPU), a digital signal processor (DSP), or other programmable logic devices, discrete gates or transistor logic devices, discrete hardware components.

The computer device 300 may also include a memory 302, wherein the processor 301 and the memory 302 are connected via a bus 303 such as an I2C (Inter-integrated Circuit) bus.

Specifically, the memory 302 may be a Flash chip, a read-only memory (ROM), a magnetic disk, an optical disc, a USB drive, or a mobile hard disk.

The processor is configured to execute the computer program stored in the memory so as to implement the ultrasound examination method provided in any embodiment of the present application when executing the computer program.

In an embodiment, the processor is used to run the computer program stored in the memory and implement the following steps when executing the computer program:
obtaining an ultrasound examination task for a patient through an ultrasound workstation system;
transmitting the ultrasound examination task to the ultrasound device;
receiving ultrasound data corresponding to the ultrasound examination task returned by the ultrasound device;
obtaining an examination result based on the ultrasound data input by a user; and
transmitting the ultrasound data and the examination result to the ultrasound workstation system.

In an embodiment, the specialized software system is a browser or a client application.

In an embodiment, the specialized software system is connected to the ultrasound workstation system via a predetermined first communication protocol or a predefined first interface defined.

In an embodiment, when the processor implements the step of obtaining the ultrasound examination task for the patient via the ultrasound workstation system, it is configured to:
when the ultrasound workstation system opens the patient report, obtain the ultrasound examination task for the patient based on the patient report.

In an embodiment, when the processor implements the step of obtaining the ultrasound examination task for the patient via the patient report, it is configured to:
capture a screenshot of the patient report;
identify a patient's identification information from the screenshot content; and
query the ultrasound examination task for the patient from a task worklist based on the identification information.

In an embodiment, when the processor implements the step of obtaining the ultrasound examination task for the patient through the patient report, it is configured to:
obtain an identification information inputted by a user, which information is displayed on the patient report;
query the ultrasound examination task for the patient from the task worklist based on the identification information.

In an embodiment, the patient report contains the patient examination information; and when the processor implements the step of obtaining the ultrasound examination task for the patient through the patient report, it is configured to:
receive the patient examination information sent by the ultrasound workstation system; and
obtain the ultrasound examination task for the patient based on the patient examination information.

In an embodiment, after the processor implements the step of receiving the ultrasound data corresponding to the ultrasound examination task returned by the ultrasound device, it is also configured to:
send the ultrasound data to a server for storage, wherein the server includes at least one of: a hospital information system server, an electronic medical record system server, an ultrasound workstation system server, and a specialized software system server.

In an embodiment, the examination result is structured data.

In an embodiment, before the processor implements the transmission of the ultrasound data and the examination result to the ultrasound workstation, it is also configured to:
analyze the examination result into text format data.

In an embodiment, the specialized software system includes an obstetric software system, and the obstetric software system provides specialized functions for ultrasound examinations in the obstetric field.

In an embodiment, the ultrasound data includes at least one of: ultrasound images, fetal measurement data, and fetal growth charts.

In an embodiment, the processor is further configured to:
transmit multiple sets of historical fetal measurement data to the ultrasound device so as to enable the ultrasound device to generate a fetal growth chart based on the multiple sets of historical fetal measurement data, wherein the fetal growth chart is used to evaluate fetal growth.

In an embodiment, the processor is further configured to:
obtain a reference table for evaluating standard fetal growth criteria; and
transmit the reference table to the ultrasound device.

In an embodiment, the processor is configured to execute a computer program stored in the memory and implements the following steps when executing the computer program:
the ultrasound workstation system opens the patient report;
the specialized software system acquires ultrasound examination task for the patient through the patient report;
the specialized software system transmits the ultrasound examination task to the ultrasound device;
the specialized software system receives the ultrasound data corresponding to the ultrasound examination task returned by the ultrasound device;
the specialized software system acquires the examination result based on the ultrasound data input by the user; and
the ultrasound workstation system receives the ultrasound data and the examination result sent by the specialized software system.

In an embodiment, the specialized software system is a browser or a client application.

In an embodiment, the specialized software system is connected to the ultrasound workstation system via a predetermined first communication protocol or a predetermined first interface.

In an embodiment, when the processor implements the acquisition of ultrasound examination task for the patient through the patient report, it is configured to:
capture a screenshot of the patient report;
identify a patient's identification information from the screenshot content; and query the ultrasound examination task for the patient from a task worklist based on the identification information.

In an embodiment, when the processor implements the acquisition of ultrasound examination task for the patient through the patient report, it is configured to:
obtain identification information inputted by a user, which information is displayed on the patient report; and
query the ultrasound examination task for the patient from the task worklist based on the identification information.

In an embodiment, the patient report contains patient examination information, and when the processor implements the acquisition of ultrasound examination task for the patient through the patient report, it is configured to:
receive the patient examination information sent by the ultrasound workstation system; and
obtain the ultrasound examination task for the patient based on the patient examination information.

In an embodiment, after the processor implements the receiving of the ultrasound data corresponding to the ultrasound examination task returned by the ultrasound device, it is further configured to:
transmit the ultrasound data to a server for storage; where the server includes at least one of the following: a hospital information system server, an electronic medical record system server, an ultrasound workstation system server, and a specialized software system server.

In an embodiment, the examination result is structured data.

In an embodiment, before the processor implements the transmission of the ultrasound data and the examination results to the ultrasound workstation system, it is further configured to:
analyze the examination results into text format data.

In an embodiment, the specialized software system includes an obstetric software system, which provides specialized functions for ultrasound examinations in the obstetric field.

In an embodiment, the ultrasound data includes at least one of ultrasound images, fetal measurement data, and fetal growth charts.

In an embodiment, the processor is further configured to:
transmit multiple sets of historical fetal measurement data by the specialized software system to the ultrasound device, so that the ultrasound device generates a fetal growth chart based on the multiple historical fetal measurement data, where the fetal growth chart is used to evaluate the fetal growth status.

In an embodiment, the processor is further configured to:
acquire a reference table for evaluating the standard fetal growth criteria by the specialized software system; and
upon the specialized software system establishing a connection with the ultrasound device, transmit the reference table to the ultrasound device.

In an embodiment, the processor is further configured to:
insert the ultrasound data and the examination results into the patient report by the ultrasound workstation system.

In an embodiment, the processor is further configured to:
upload the patient report to the electronic medical record system by the ultrasound workstation system when the patient report has been approved.

It should be understood by those skilled in the art that the structure shown in FIG. 7 is merely a block diagram of some of the components related to the embodiments of the present application and does not constitute a limitation on the computer device. The specific computer device may include more or fewer components than those shown in the figure, or combine some components, or have a different configuration of components.

It should be noted that those skilled in the art can clearly understand that for brevity and clarity, the specific working process of the computer device described above may be understood by reference to the corresponding process in the aforementioned embodiments of the ultrasound examination method, and will not be repeated herein.

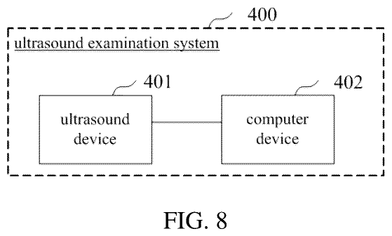

Please refer to FIG. 8, which is a schematic block diagram of an ultrasound examination system provided in some embodiments of the present application.

As shown in FIG. 8, the ultrasound examination system 400 includes an ultrasound device 401 and a computer device 402, wherein the computer device 402 is connected to the ultrasound device 401.

The computer device 402 may include the computer device 300 shown in FIG. 7. For example, the computer device 402 includes a specialized software system and an ultrasound workstation system, wherein the specialized software system provides specialized functions for ultrasound examinations in a specific field; and the specialized software system is connected to the ultrasound workstation system in the form of middleware.

In an embodiment, the computer device 402 is connected to the ultrasound device 401 via a predetermined second communication protocol or a predefined second interface. The second interface may include, but is not limited to, an application programming interface (API) and network sockets. The second communication protocol may include, but is not limited to, the digital imaging and communications in medicine (DICOM) protocol and a private communication protocol.

It should be noted that those skilled in the art can clearly understand that for brevity and clarity, the specific working process of the ultrasound examination system described above may be understood by reference to the corresponding process in the embodiments of the ultrasound examination method provided herein, and will not be repeated here.

The embodiments of the present application further provides a computer-readable storage medium having stored thereon one or more programs executable by one or more processors to cause the processor(s) to perform the steps of any ultrasound examination method provided in the embodiments herein.

The storage medium may be an internal storage of the computer device described in the foregoing embodiments, for example, a hard disk or memory of the computer device. Alternatively, the storage medium may be an external storage device associated with the computer device, such as a plug-in hard disk, a smart media card (SMC), a secure digital (SD) card, or a flash card provided on the computer device.

Those skilled in the art will appreciate that all or some steps in the methods disclosed herein, and functional modules/units in the systems and devices may be implemented as software, firmware, hardware, or any suitable combination thereof. In hardware implementations, the division of functional modules/units described above need to correspond to physical hardware partitioning; for instance, a single hardware component may perform multiple functions, or a particular function or step may be cooperatively executed by several hardware components. Some or all hardware components may be implemented as: software executed by a processor (such as a central processing unit, a digital signal processor, or a microprocessor), or dedicated hardware circuitry, or integrated circuits such as application-specific integrated circuits. Such software may be distributed on computer-readable media, comprising: non-transitory computer storage media, and transitory communication media.

Computer storage media include, but are not limited to, RAM, ROM, EEPROM, flash memory, or other memory technologies, CD-ROM, digital versatile disks (DVD), or other optical disk storage, magnetic cassettes, magnetic tapes, magnetic disk storage, or other magnetic storage devices, or any other medium capable of storing desired information that is accessible by a computer. In addition, communication media typically embody computer-readable instructions, data structures, program modules, or other data in modulated data signals, such as carrier waves or other transmission mechanisms, and can include any information delivery medium.

The serial numbers of the embodiments of the present application mentioned above serves only for description and do not imply any preference among embodiments. The foregoing implementations are merely illustrative of the present application, whose protection scope shall extend to any equivalent modifications or substitutions conceivable by those skilled in the art within the technical scope disclosed herein. Such modifications and substitutions shall fall within the protection scope of the present application, which shall be determined solely by the appended claims.

1. An ultrasound examination method, adapted for use with a specialized software system, wherein the specialized software system provides specialized functions for specialized ultrasound examinations, and is connected to an ultrasound workstation system in a form of middleware; the method comprising:
obtaining an ultrasound examination task for a patient through the ultrasound workstation system;
transmitting the ultrasound examination task to an ultrasound device;
receiving ultrasound data corresponding to the ultrasound examination task from the ultrasound device;
obtaining an examination result that is inputted by a user based on the ultrasound data; and
sending the ultrasound data and the examination result to the ultrasound workstation system.

2. The ultrasound examination method according to claim 1, wherein the specialized software system is a browser or a client application.

3. The ultrasound examination method according to claim 1, wherein the specialized software system is connected to the ultrasound workstation system via a predetermined first communication protocol or a predefined first interface.

4. The ultrasound examination method according to claim 1, wherein obtaining an ultrasound examination task for a patient through the ultrasound workstation system, comprises:
when opening a patient report in the ultrasound workstation system, obtaining the ultrasound examination task for the patient based on the patient report.

5. The ultrasound examination method according to claim 4, wherein obtaining the ultrasound examination task for the patient based on the patient report, comprises:
capturing a screenshot of the patient report;
identifying identification information about the patient from a content of the screenshot; and
querying the ultrasound examination task for the patient from a task worklist based on the identification information.

6. The ultrasound examination method according to claim 4, wherein obtaining the ultrasound examination task for the patient based on the patient report, comprises:
obtaining identification information inputted by a user, which information is displayed on the patient report; and
querying the ultrasound examination task for the patient from a task worklist based on the identification information.

7. The ultrasound examination method according to claim 1, further comprising:
obtaining patient information or historical examination data of the patient through the ultrasound workstation system; and
transmitting the patient information or the historical examination data to the ultrasound device.

8. The ultrasound examination method according to claim 1, wherein the specialized software system comprises an obstetric software system, and the obstetric software system provides specialized functions for ultrasound examinations in an obstetric specialty field.

9. The ultrasound examination method according to claim 8, wherein the ultrasound data comprises at least one of: an ultrasound image, fetal measurement data, and a fetal growth chart.

10. The ultrasound examination method according to claim 8, further comprising:
transmitting multiple sets of historical fetal measurement data to the ultrasound device, so that the ultrasound device generates a fetal growth chart according to the multiple sets of historical fetal measurement data, wherein the fetal growth chart is used to evaluate fetal growth.

11. The ultrasound examination method according to claim 8, further comprising:
obtaining a reference table for evaluating standard fetal growth criteria; and
transmitting the reference table to the ultrasound device.

12. An ultrasound examination method, adapted for use with a computer device, wherein the computer device comprises a specialized software system and an ultrasound workstation system, the specialized software system provides specialized functions for specialized ultrasound examinations, and the specialized software system is connected to the ultrasound workstation system in a form of middleware; the method comprising:
the ultrasound workstation system opening a patient report;
the specialized software system obtaining an ultrasound examination task for a patient based on the patient report;
the specialized software system transmitting the ultrasound examination task to an ultrasound device;
the specialized software system receiving ultrasound data that corresponds to the ultrasound examination task from the ultrasound device;
the specialized software system obtaining an examination result that is inputted by a user based on the ultrasound data;
the ultrasound workstation system receiving the ultrasound data and the examination result that is sent by the specialized software system.

13. The ultrasound examination method according to claim 12, wherein the specialized software system obtaining an ultrasound examination task for a patient based on the patient report, comprises:
the specialized software system capturing a screenshot of the patient report;
the specialized software system identifying identification information about the patient from a content of the screenshot; and
the specialized software system querying the ultrasound examination task for the patient from a task worklist based on the identification information.

14. The ultrasound examination method according to claim 12, wherein the specialized software system obtaining an ultrasound examination task for a patient based on the patient report, comprises:
the specialized software system obtaining identification information inputted by a user, which information is displayed on the patient report; and
the specialized software system querying the ultrasound examination task for the patient from a task worklist based on the identification information.

15. The ultrasound examination method according to claim 12, further comprising:
the specialized software system obtaining patient information or historical examination data of the patient through the ultrasound workstation system; and
the specialized software system transmitting the patient information or the historical examination data to the ultrasound device.

16. The ultrasound examination method according to claim 12, wherein the specialized software system comprises an obstetric software system, and the obstetric software system provides specialized functions for ultrasound examinations in an obstetric specialty field.

17. The ultrasound examination method according to claim 16, wherein the ultrasound data comprises at least one of: an ultrasound image, fetal measurement data, and a fetal growth chart.

18. The ultrasound examination method according to claim 16, further comprising:
the specialized software system transmitting multiple sets of historical fetal measurement data to the ultrasound device, so that the ultrasound device generates a fetal growth chart according to the multiple sets of historical fetal measurement data, wherein the fetal growth chart is used to evaluate fetal growth.

19. The ultrasound examination method according to claim 16, further comprising:
the specialized software system obtaining a reference table for evaluating standard fetal growth criteria; and
the specialized software system transmitting the reference table to the ultrasound device while connected to the ultrasound device.

20. The ultrasound examination method according to claim 12, further comprising:
the ultrasound workstation system inserting the ultrasound data and the examination result into the patient report; and
the ultrasound workstation system uploading the patient report to an electronic medical record system when the patient report has been approved.
